(19) United States
(12) Patent Application Publication
(10) Pub. No.: US 2011/0190745 A1
(43) Pub. Date: Aug. 4, 2011

(54) TREATMENT OF SWEAT GLANDS

(76) Inventors: Nathan S. Uebelhoer, San Diego, CA (US); Peter Shumaker, San Diego, CA (US); Yacov Domankevitz, Zichron-Yaacov (IL)

(21) Appl. No.: 12/960,191

(22) Filed: Dec. 3, 2010

Related U.S. Application Data
(60) Provisional application No. 61/266,849, filed on Dec. 4, 2009.

(51) Int. Cl. A61B 18/18

(52) U.S. Cl. 606/9

(57) ABSTRACT

A treatment of sweat glands in a target region of skin includes generating electromagnetic radiation having a wavelength of about 1,064 nm to about 1,800 nm. To decrease sweat production in a plurality of sweat glands, the electromagnetic radiation is delivered to a dermal interface defined by a dermal region and a subcutaneous fat region in the target region of skin to cause thermal injury to at least one of the dermal region, the subcutaneous fat region or the dermal interface. An epidermal region of the skin can be cooled at least one of before, during or after delivering the electromagnetic radiation to the dermal interface in the target region of skin.

### CROSS REFERENCE TO RELATED APPLICATIONS

This application claims the benefit of and priority to U.S. Provisional Application Ser. No. 61/266,849 filed Dec. 4, 2009, which is owned by the assignee of the instant application and the disclosure of which is incorporated herein by reference in its entirety.

### FIELD OF THE INVENTION

The invention relates generally to the treatment of sweat glands and sweat-related conditions caused by increased eccrine and/or apocrine sweat gland production, and more particularly, to causing thermal injury in the epidermal and/or dermal region of skin that includes eccrine and/or apocrine glands.

### BACKGROUND OF THE INVENTION

Hyperhidrosis is a medical condition in which a person sweats excessively and unpredictably. Individuals with hyperhidrosis may sweat even when the temperature is cool or when they are at rest.

Bromhidrosis, also known as bromidrosis or body odor, is a common phenomenon in postpubertal individuals. Bromhidrosis can be a chronic condition in which excessive odor emanates from the skin. Bromhidrosis usually results from apocrine gland secretion.

Hidradenitis suppurativa (HS) is a skin disease that most commonly affects areas bearing apocrine sweat glands or sebaceous glands, such as the underarms, breasts, inner thighs, groin and buttocks. HS manifests as clusters of chronic abscesses, epidermoid cyst, sebaceous cysts, pilonidal cyst or multilocalised infections, which can be as large as baseballs or as small as a pea, that are extremely painful to the touch and may persist for years with occasional to frequent periods of inflammation, culminating in incision and drainage of pus, often leaving open wounds that will not heal.

Eccrine glands begin to form during the fourth month of gestation as a downgrowth of the epidermis known as the eccrine germ. They first develop on the palms and soles, and then gradually appear on the remainder of the body, with the exception of the vermilion border of the lips, nail beds, labia minora, glans penis, and inner surface of the prepuce. The highest density of eccrine glands is seen on the palms, soles, and axillae.

As shown in FIG. 1, the eccrine germ later forms the three portions of the eccrine gland 100: the intraepidermal portion (acrosyringium) 104, the intradermal duct (coiled duct 106a and straight duct 106b), and the secretory portion (coiled gland) 108. The coiled gland 108 is located in the deep dermis or at the border of the dermis and subcutaneous fat. The coiled gland 108 is composed of one distinct layer of clear and dark cells. The clear cells secrete glycogen, water, and electrolytes, and the dark cells secrete sialomucin. These secretory cells are surrounded by contractile myoepithelial cells enclosed within a hyaline basement membrane with peripheral collagen fibers.

The eccrine duct extends upward from the coiled gland 108 through the dermis, first as the coiled duct 106b and then as the straight duct 106a. The eccrine duct is composed of an outer layer of basal cells, and an inner layer of cells whose luminal surface forms an eosinophilic cuticle. The straight duct 106a ends as it enters into a wide rete ridge of the epidermis, also known as an eccrine sweat duct ridge. The duct is now referred to as the acrosyringium 104 as it spirals through the epidermis and opens directly onto the skin surface.

Eccrine sweat is produced via merocrine secretion in the coiled gland 108, and is composed of water, sodium, potassium lactate, urea, ammonia, serine, ornithine, citrulline, aspartic acid, heavy metals, organic compounds, and proteolytic enzymes. Acetylcholine production results in an increased calcium level in the secretory cell cytoplasm, followed by an intricate series of sodium, potassium, and chloride ion exchanges, which ultimately lead to sodium and chloride movement into the gland lumen. The initially isotonic eccrine sweat then travels through the eccrine duct where NaCl and HCO₃ are actively reabsorbed. Only about 25 percent of the sodium can be reabsorbed. Eccrine sweat then passes through the acrosyringium 104 and is deposited on the skin's surface.

The primary function of the eccrine unit 100 is thermoregulation, which is accomplished through the cooling effects of evaporation of this sweat on the skin's surface. Stimulation of eccrine sweat production is mediated predominantly through postganglionic C fiber production of acetylcholine. Emotional stressors tend to induce sweating that is confined mainly to the palms and soles. All eccrine units 100 can be utilized to respond to the body's changing thermoregulatory needs.

Apocrine glands 110, as shown in FIG. 2, are outgrowths of the superior portions of pilosebaceous units 112 (hair follicles). The pilosebaceous unit 112 forms throughout the fourth to sixth month, initially on the scalp, and then on the remainder of the skin's surface. A primary epithelial germ (hair germ) grows down from the epidermis and forms an apocrine gland, sebaceous gland, and hair follicle.

Apocrine glands 110 include: (1) a coiled gland 114 in the deep dermis or at the junction of the dermis and subcutaneous fat; and (2) a straight duct 118 that traverses the dermis and empties into the isthmus (uppermost portion) of a hair follicle. The coiled gland 114 includes one layer of secretory cells around a lumen that is about 10 times the diameter of its eccrine counterpart. Contractile myoepithelial cells, a hyaline basement membrane, and connective tissue surround the coiled gland 114. The straight duct 118 runs from the coiled gland 114 to the isthmus of the hair follicle and is identical in appearance to the eccrine straight duct, with two cuboidal layers of cells encircling a lumen lined with an eosinophillic cuticle.

The predominant mode of apocrine secretion is decapitation, a process where the apical portion of the secretory cell cytoplasm pinches off and enters the lumen of the gland. Apocrine sweat consists mainly of sialomucin. Although odorless initially, as apocrine sweat comes in contact with normal bacterial flora on the surface of the skin, an odor develops. Apocrine sweat is more viscous and produced in much smaller amounts than eccrine sweat, (which actually is the wet portion of axillary sweat). The exact function of apocrine glands 110 is unclear, although they are thought to represent scent glands.

At the time of birth, apocrine glands 110 are present primarily in the axillae and anogenital regions, where they remain small and nonfunctional until puberty. Specialized variants of apocrine glands 110 also exist: the Moll's glands seen on the eyelids; the cerumen-producing (ear wax) glands of the external auditory canal; and the milk-producing glands of the breasts. At puberty, hormonal stimulation causes apocrine glands 110 to become functional, and they respond mainly to sympathetic adrenergic stimuli initiated by emotional stressors. Also during puberty, apocrine glands 110 appear. They are hybrid sweat glands that are found in the axilla. Apocrine glands 110 might play a role in axillary hyperhidrosis. Their secretory glands have both a small-diameter portion similar to an eccrine gland 100 and a large-diameter portion that resembles an apocrine gland 110. Similar to eccrine glands 100, apocrine glands 110 respond mainly to cholinergic stimuli, and their ducts are long and open directly onto the skin's surface. However, apocrine glands 110 secrete nearly 10 times as much sweat as eccrine glands 100.

A variety of diseases are caused by or lead to abnormal function of the eccrine glands 100 and apocrine glands 110. Hyperhidrosis, or excessive eccrine sweat secretion, can be generalized in certain systemic, central nervous system, or peripheral nervous system diseases. Localized hyperhidrosis of the palms and soles is often due to emotional stressors. Varying degrees of hyperthermia are associated with decreased eccrine sweating (hypohidrosis) or absent sweating (anhidrosis) in hereditary disorders such as the ectodermal dysplasias. Hypohidrotic ectodermal dysplasia, also known as Christ-Siemens-Touraine syndrome, is an X-linked recessive disease that consists of the triad of hypotrichosis, anodontia, and hypohidrosis, along with characteristic facies. Hidrotic ectodermal dysplasia, also known as Clouston's syndrome, is an autosomal dominant disorder with normal facial features and active eccrine glands, alopecia, nail dystrophy, eye changes, and palmoplantar hyperkeratosis. Hypohidrosis or anhidrosis can also be seen in acquired conditions like heat stroke or heat exhaustion. Patients with cystic fibrosis are unable to reabsorb sodium in the eccrine duct, and therefore they have elevated sodium concentrations in their eccrine sweat. A uremic frost, seen on the skin of patients with severe renal failure and markedly elevated serum urea levels, is the result of increased urea and salt deposits from the eccrine sweat.

Excessive heat and humidity that causes profuse eccrine sweating might sometimes be accompanied by blockage of the eccrine units 100 with subsequent duct rupture and varying degrees of inflammation presenting as miliaria. If ductal obstruction occurs within the stratum corneum, then miliaria crystallina develops with asymptomatic superficial vesicles and no surrounding inflammation. When ductal obstruction is found deeper in the epidermis, then miliaria rubra (prickly heat) appears as pruritic or tender red macules or papules, which are often located on the thorax and neck. Prolonged exposure to tropical environments resulting in multiple episodes of miliaria rubra can lead to the development of miliaria profunda, with asymptomatic skin-colored papules forming as a result of eccrine duct obstruction at or below the dermoepidermal junction. Fox-Fordyce disease, or apocrine miliaria, develops when minor inflammation follows intraepidermal rupture of apocrine ducts. More intense inflammation due to follicular obstruction can secondarily involve the apocrine units 110 in hidradenitis suppurativa. Multiple benign and malignant neoplasms also can originate in both eccrine glands 100 and apocrine glands 110, the most common being benign syringomata on the lower eyelids of adult women.

### SUMMARY OF THE INVENTION

The invention, in various embodiments, features treatments of eccrine and apocrine glands. The treatments are with fewer side effects, lower cost, and less risk than prior art treatments. Instead of being invasive surgical procedures, radiation is directed through the surface of the skin. Longer, lasting benefits than prior art treatments can be achieved.

Sweat-related conditions that can be treated using the technology include, but are not limited to, hyperhidrosis, bromhidrosis, hidradenitis suppurativa, hypohidrotic ectodermal dysplasia, hypotrichosis, anodontia, hypohidrosis, hidrotic ectodermal dysplasia, uremic frost, neoplasms, syringomata, and various forms of miliarias.

In one aspect, the invention features a method of treating sweat glands in a target region of the skin. Electromagnetic radiation is generated having a wavelength of about 700 nm to about 1,800 nm, and delivered to a dermal interface defined by a dermal region and a subcutaneous fat region of the target region of skin. The treatment can cause thermal injury to the dermal region, the subcutaneous fat region and/or the dermal interface to decrease sweat production in a plurality of sweat glands. An epidermal region of the skin can be cooled at least one of before, during or after delivering the electromagnetic radiation to the dermal interface in the target region of skin.

In another aspect, the invention features a method of treating sweat glands in a target region of skin. Electromagnetic radiation is generated having a wavelength of about 700 nm to about 1,800 nm. The electromagnetic radiation is delivered to hair bulbs in communication with at least some of the sweat glands in the target region of skin to cause thermal injury to the hair bulbs. Decreased sweat production in the at least some of the sweat gland is induced. In certain embodiments, the electromagnetic radiation is delivered to a dermal interface defined by a dermal region and a subcutaneous fat region in the target region of skin. An epidermal region of the skin can be cooled at least one of before, during or after delivering the electromagnetic radiation to the dermal interface in the target region of skin.

In yet another aspect, the invention features an apparatus for treating sweat glands in a target region of skin. The apparatus includes a source for generating electromagnetic radiation having a wavelength of about 700 nm to about 1,800 nm. The apparatus also includes a delivery system coupled to the source for directing the electromagnetic radiation to a dermal interface defined by a dermal region and a subcutaneous fat region in the target region of skin. The electromagnetic radiation can cause thermal injury to the dermal region, the subcutaneous fat region and/or the dermal interface to decrease sweat production in a plurality of sweat glands. A cooling system can cool an epidermal region of the skin at least one of before, during or after delivering the electromagnetic radiation to the dermal interface in the target region of skin.

In still another aspect, the invention features an apparatus for treating a sweat gland in a target region of skin. The apparatus includes a source for generating electromagnetic radiation having a wavelength of about 1,160 nm to about 1,800 nm. The apparatus also includes a delivery system coupled to the source for directing the electromagnetic radiation to hair bulbs in communication with at least some of the sweat glands in the target region of skin to cause thermal injury to the hair bulb. Decreased sweat production of the sweat gland is induced. A cooling system can cool an epidermal region of the skin at least one of before, during or after delivering the electromagnetic radiation to the dermal interface in the target region of skin.

In another aspect, the invention features an apparatus for treating sweat glands in a target region of the skin. The apparatus includes means for generating electromagnetic radiation having a wavelength of about 700 nm to about 1,800 nm, means for delivering the electromagnetic radiation to a dermal interface defined by a dermal region and a subcutaneous fat region of the target region of skin, means for cooling an epidermal region of the skin at least one of before, during or after delivering the electromagnetic radiation to the dermal interface in the target region of skin, and means for causing thermal injury to the dermal region, the subcutaneous fat region and/or the dermal interface to decrease sweat production in a plurality of sweat glands.

In other examples, any of the aspects above, or any apparatus, system or device, or method, process or technique, described herein, can include one or more of the following features.

In certain embodiments, the sweat gland is thermally injured. In certain embodiments, a hair bulb in communication with the sweat gland is thermally injured. In certain embodiments, the sweat gland is destroyed. In certain embodiments, the treatment causes sufficient thermal injury to induce collagen formation to strength the target region of skin. In certain embodiments, the treatment causes sufficient thermal injury to induce fibrosis in the dermal region, the subcutaneous fat region, and/or the dermal interface.

In various embodiments, the wavelength of the electromagnetic radiation is about 1,190 nm to about 1,230 nm (e.g., 1,210 nm). In some embodiments, the wavelength is about 1,064 nm. In various embodiments, the electromagnetic radiation has a fluence of about 30 J/cm² to about 300 J/cm² (e.g., 50-60 J/cm²). The electromagnetic radiation can have a pulse duration of about 1 second to about 10 seconds.

In various embodiments, the radiation is delivered to the target region about 0.5 mm to about 5 mm below the surface of the skin. In various embodiments, the radiation is delivered to cause treatment temperature to peak at the dermal interface.

In various embodiments, cooling can be contact cooling, air cooling, or cryogen spray cooling. In certain embodiments, the target region of skin is massaged before, during, and/or after irradiation.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows a sectional view of skin including an eccrine gland.

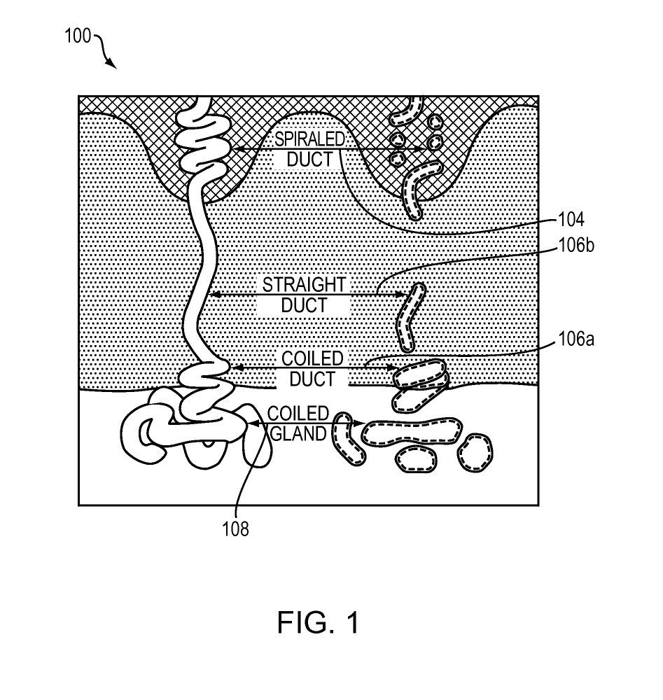

FIG. 2 shows a sectional view of skin including an apocrine gland.

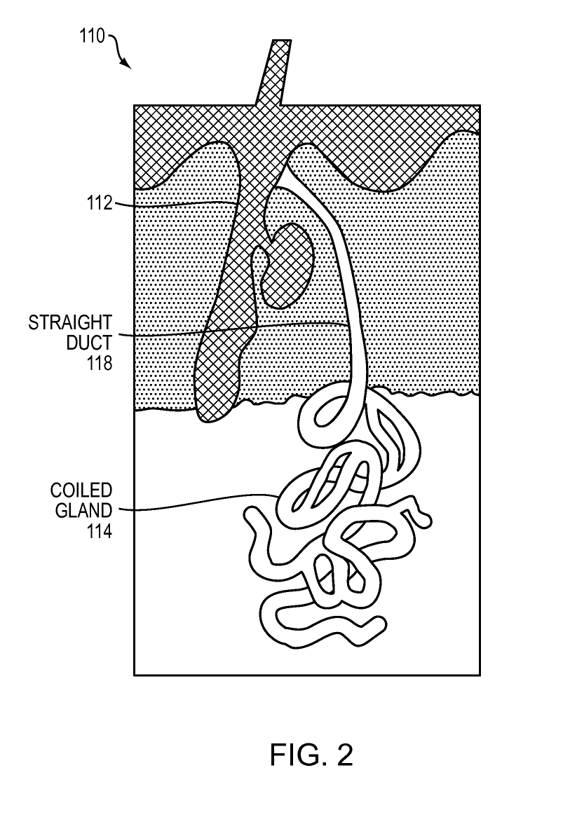

FIG. 3 shows a sectional view of skin being treated by a beam of radiation.

FIG. 4 shows an exemplary system for treating eccrine and/or apocrine glands.

FIG. 5 shows a planoconvex lens positioned on a skin surface.

FIG. 6 shows a plurality of lens focusing radiation to a target region of skin.

FIG. 7 shows a lens having a concave surface positioned on a skin surface.

FIG. 8 shows an exemplary handpiece having rollers to massage skin.

### DETAILED DESCRIPTION OF THE INVENTION

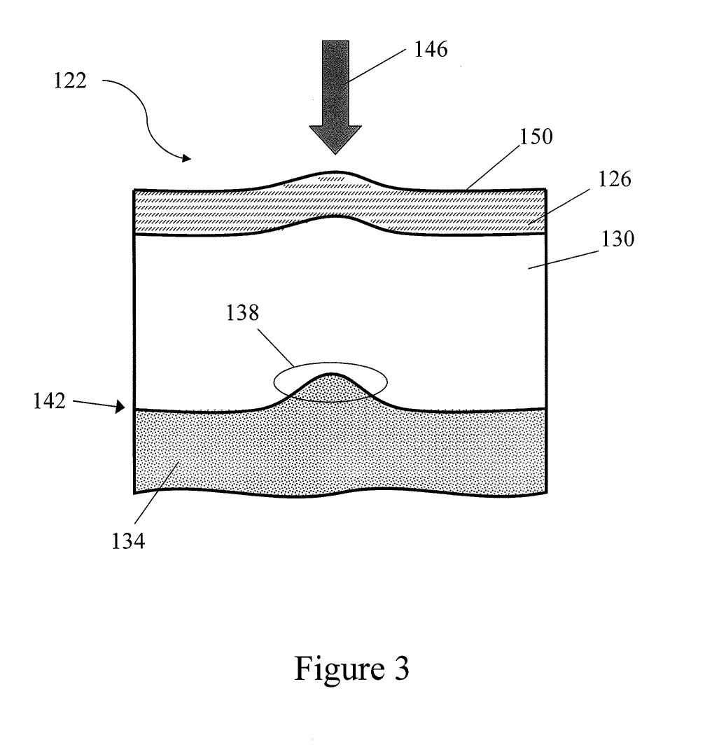

FIG. 3 shows a cross-section of skin 122 including an epidermal layer 126, a dermal layer 130, and a layer of fatty tissue 134. In certain embodiments, functions of a sweat gland can be affected by targeting a dermal-subcutaneous interface region 138, at or about the dermal interface 142. The dermal-subcutaneous interface region 138 can include the gland or a hair bulb connected to the gland. A beam of radiation 146 can be used to cause thermal injury to the target region 138 by delivery through a surface 150 of the epidermal layer 126. In operation, the radiation 146 can penetrate through the epidermal layer 126 and the dermal layer 130, and can treat one or more of a portion of the dermal layer 130, a portion of the layer of fatty tissue 134, a gland disposed in the target region 138, or a hair bulb disposed in the target region 138. The target region 138 can be in one or both of the dermal layer 130 or the layer of fatty tissue 134. The beam of radiation 146 can be delivered to the target region 138 to thermally injure, damage, and/or destroy the gland and/or the hair bulb.

In various embodiments, a zone of thermal injury can be formed in, at or proximate to the dermal interface 142. Fatty tissue has a specific heat that is lower than that of surrounding tissue. For example, fatty tissue has a volumetric specific heat of about 1.8 J/cm³ K, whereas skin has a volumetric specific heat of about 4.3 J/cm³ K. In one embodiment, the peak temperature of the tissue can be caused to form in, at or proximate to the dermal interface 142. For example, a predetermined wavelength, fluence, pulse duration, and cooling parameters can be selected to position the peak of the zone of thermal injury in, at or proximate to the dermal interface 142. In certain embodiments, the gland and/or hair bulb is heated by absorption of radiation, and heat can be conducted into dermal tissue or fatty tissue proximate to the dermal interface 142.

The radiation 146 for treating eccrine and/or apocrine glands can treat another indication simultaneously or substantially simultaneously, or the radiation 146 can be combined with another source of radiation to treat another indication simultaneously or substantially simultaneously. For example, subcutaneous fat and/or cellulite can be treated by injuring fatty tissue (e.g., a fatty deposit located at or proximate to the dermal interface 142) and by thickening and/or strengthening of the dermis, which can prevent and/or preclude additional fatty tissue from perturbing the dermal interface 142. In various embodiments, a treatment can, for example, reduce fat, remove a portion of fat, improve skin laxity, tighten skin, strengthen skin, thicken skin, induce new collagen formation, promote fibrosis of the dermal layer or subcutaneous fat layer, or be used for a combination of the aforementioned.

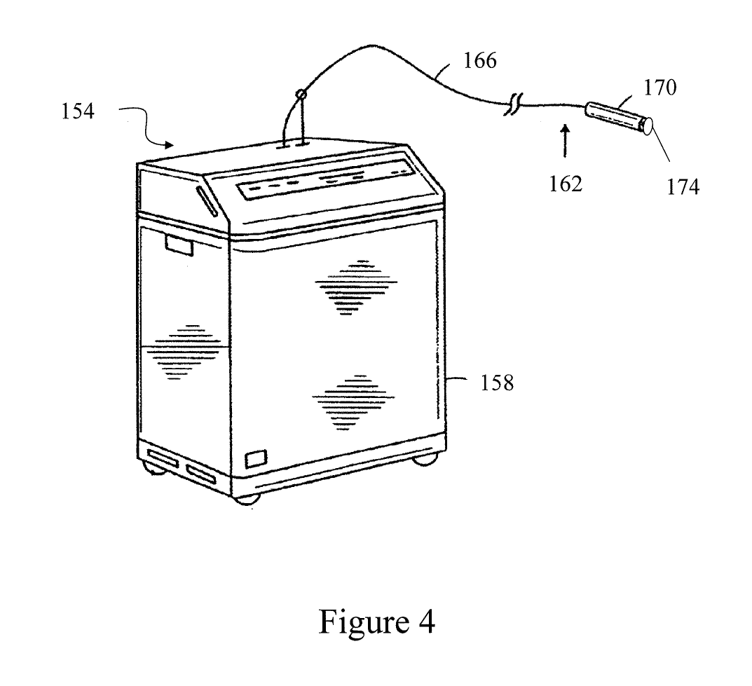

FIG. 4 shows an exemplary embodiment of a system 154 for treating eccrine and/or apocrine glands. The system 154 can be used to non-invasively deliver a beam of radiation to a target region. For example, the beam of radiation can be delivered through an external surface of skin over a target region. The system 154 includes an energy source 158 and a delivery system 162. In one embodiment, a beam of radiation provided by the energy source 158 is directed via the delivery system 162 to the target region. In the illustrated embodiment, the delivery system 162 includes a fiber 166 having a circular cross-section and a handpiece 170. A beam of radiation can be delivered by the fiber 166 to the handpiece 170, which can include an optical system (e.g., an optic or system of optics) to direct the beam of radiation to the target region. A user can hold or manipulate the handpiece 170 to irradiate the target region. The delivery system 162 can be positioned in contact with a skin surface, can be positioned adjacent a skin surface, can be positioned proximate a skin surface, can be positioned spaced from a skin surface, or a combination of the aforementioned. In the embodiment shown, the delivery system 162 includes a spacer 174 to space the delivery system 162 from the skin surface. In one embodiment, the spacer 174 can be a distance gauge, which can aid a practitioner with placement of the delivery system 162.

In various embodiments, the energy source 158 can be an incoherent light source, a coherent light source (e.g., a laser), a microwave generator, or a radio-frequency generator. In one embodiment, the source generates ultrasonic energy that is used to treat the tissue. In some embodiments, two or more sources can be used together to effect a treatment. For example, an incoherent source can be used to provide a first beam of radiation while a coherent source provides a second beam of radiation. The first and second beams of radiation can share a common wavelength or can have different wavelengths. In an embodiment using an incoherent light source or a coherent light source, the beam of radiation can be a pulsed beam, a scanned beam (e.g., a scanned continuous wave (CW) beam), or a gated CW beam.

The incoherent light source can be an intense pulsed light system, a fluorescent pulsed light system, a lamp or flashlamp, or a light emitting diode system. The coherent light source can be a laser. For example, the laser can be, but is not limited to, a pulsed dye laser, a Nd:YAG laser, a frequency doubled Nd:YAG laser, a Nd:glass laser, a copper vapor laser, an alexandrite laser, a frequency doubled alexandrite laser, a titanium sapphire laser, a ruby laser, a fiber laser, or a diode laser. Exemplary laser systems are the GentleLase, GentleYAG, GentleMax, and AlexTriVantage available from Candela Corporation (Wayland, Mass.).

In various embodiments, the beam of radiation can have a wavelength of about 400 nm to about 2,600 nm, although longer and shorter wavelengths can be used depending on the application. In some embodiments, the wavelength is about 700 nm to about 1,800 nm. In various embodiments, the wavelength radiation is about 1,190 nm to about 1,230 nm. The wavelength can be about 1,064 nm or about 1,210 nm.

In various embodiments, the beam of radiation can have a fluence of about 1 J/cm² to about 500 J/cm², although higher and lower fluences can be used depending on the application. In some embodiments, the fluence is about 10 J/cm² to about 350 J/cm². In some embodiments, the fluence is about 30 J/cm² to about 300 J/cm². The fluence can be about 50 J/cm² to about 60 J/cm².

In various embodiments, the beam of radiation can have a spotsize of about 0.5 mm to about 25 mm, although larger and smaller spotsizes can be used depending on the application. The spotsize can be about 8 to 18 mm. In one embodiment, the fluence can be from 10 to 70 J/cm² with a 12 mm spot.

In various embodiments, the beam of radiation can have a pulse duration between about 10 μs and about 30 s, although larger and smaller pulse durations can be used depending on the application. In some embodiments, the pulse duration is about 0.1 second to about 20 seconds. The pulse duration can be about 1 second and about 10 seconds. In certain embodiments, the beam of radiation can be delivered in a series of sub-pulses spaced in time such that within a region of tissue, the tissue is exposed to radiation intermittently over total time interval of about 0.1 second to about 20 seconds or about 1 second to 10 seconds.

In various embodiments, the beam of radiation can be delivered at a rate of between about 0.1 pulse per second and about 10 pulses per second, although faster and slower pulse rates can be used depending on the application.

In one embodiment, the wavelength is about 1064 nm, the pulse duration is about 3 milliseconds, the fluence range can be about 50 to 60 J/cm2, and the spot size can be about 8 to 18 mm. In certain embodiments, a CW laser with a scanner can be used. The electromagnetic radiation can be scanned over the surface of the skin in the treatment area such that the laser energy is delivered to a region of skin over an effective time period of about 0.1 second to about 20 seconds.

In certain embodiments, a CW or a repetitively pulsed laser with a scanner can be used. The scanning can also be done manually by moving the treatment beam over the treatment area. The electromagnetic radiation can be scanned over the surface of the skin in the treatment area such that the laser energy is delivered to a region of skin over an effective time period of about 0.1 second to about 20 seconds.

In various embodiments, the parameters of the radiation can be selected to deliver the beam of radiation to a predetermined depth, such as at or proximate to the dermal interface 142, as shown in FIG. 3. In some embodiments, the beam of radiation can be delivered to the target region about 0.5 mm to about 10 mm below an exposed surface of the skin, although shallower or deeper depths can be selected depending on the application. In one embodiment, the beam of radiation is delivered to the target region about 0.5 mm to about 5 mm below an exposed surface of the skin.

In various embodiments, the tissue can be heated to a temperature of between about 35° C. and about 80° C., although higher and lower temperatures can be used depending on the application. In one embodiment, the temperature is between about 38° C. and about 70° C. In one embodiment, the peak temperature of tissue can be caused to form at or proximate to the dermal interface 142, as shown in FIG. 3.

To minimize unwanted thermal injury to tissue not targeted (e.g., an exposed surface of the target region and/or the epidermal layer), the delivery system 162 shown in FIG. 4 can include a cooling system for cooling before, during or after delivery of radiation, or a combination of the aforementioned. Cooling can include contact conduction cooling, evaporative spray cooling, convective air flow cooling, or a combination of the aforementioned.

In one embodiment, the handpiece 170 includes a skin contacting portion that can be brought into contact with the skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

A spray cooling device can use cryogen, water, or air as a coolant. In one embodiment, a dynamic cooling device can be used to cool the skin (e.g., a DCD available from Candela Corporation). For example, the delivery system 162 can include tubing for delivering a cooling fluid to the handpiece 170. The tubing can be connected to a container of a low boiling point fluid, and the handpiece 170 can include a valve for delivering a spurt of the fluid to the skin. Heat can be extracted from the skin by virtue of evaporative cooling of the low boiling point fluid. The fluid can be a non-toxic substance with high vapor pressure at normal body temperature, such as a Freon, tetrafluoroethane, or liquefied CO₂.

In various embodiments, a topical osmotic agent is applied to the region of skin to be treated, prior to treatment. The osmotic agent reduces the water content in the dermis overlying the dermal interface 142, as shown in FIG. 3. This reduction in the water content can increase the transmission of the radiation into the dermal interface region and into the subcutaneous fat, thereby more effectively treating the area, reducing injury to the dermis, and reducing treatment pain. The osmotic agent can be glycerin or glycerol. A module can be used to apply the osmotic agent. The module can be a needle or syringe. The module can include a reservoir for retaining the osmotic agent and an injector for applying the agent to a skin region.

In various embodiments, a delivery system, such as the delivery system 162 shown in FIG. 4, can include a focusing system for focusing the beam of radiation below the surface of the skin in the target region. The focusing system can direct the beam of radiation to the target region about 0.1 mm to about 10 mm below the exposed surface of the skin. In some embodiments, the delivery system 162 can include a lens, a planoconvex lens, or a plurality of lens to focus the beam of radiation.

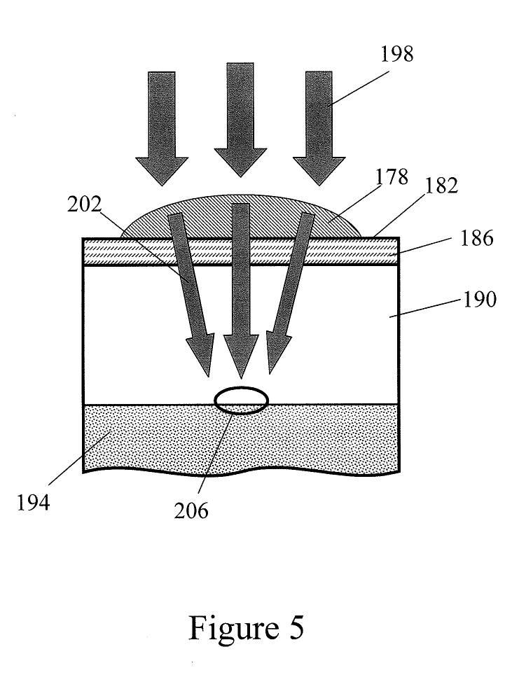

FIG. 5 shows a planoconvex lens 178 positioned on a surface 182 of a section of skin, including an epidermal region 186, a dermal region 190, and a layer of fatty tissue 194. The planoconvex lens 178 can focus radiation 198 (focusing shown by arrows 202) to a sub surface focal region 206, which can include a gland and/or hair bulb. In certain embodiments, the element contacting the skin can be pressed into or against the skin to displace blood in the dermis, thereby increasing the transmission of the radiation through the dermis and reducing unwanted injury to the skin.

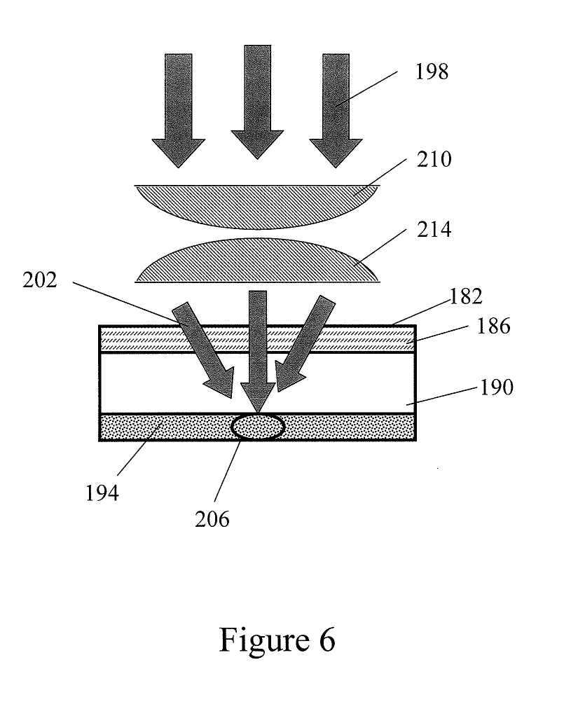

FIG. 6 shows a plurality of lens 210, 214 spaced from the skin surface 182. The plurality of lens 210, 214 can focus the radiation 198 (focusing shown by the arrows 202) to the sub surface focal region 206.

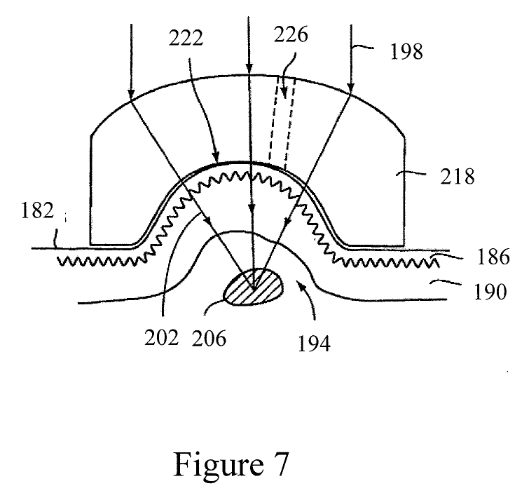

FIG. 7 shows a lens 218 having a concave surface 222 for contacting the skin surface 182. In certain embodiments, the lens 218 is placed proximate to a target region of skin. Vacuum can be applied to draw the target region of skin against the concave surface 222 of the lens 218. Vacuum can be applied through orifice 226 in the lens 218 by a vacuum device. The lens 218 can focus the radiation 198 to the sub surface focal region 206.

In various embodiments, the source of radiation can be a diode laser having sufficient power to affect one or more fat cells. An advantage of diode lasers is that they can be fabricated at specific wavelengths that target fatty tissue. A limitation, though, of many diode laser devices and solid state devices targeting fatty tissue is the inability to produce sufficient power to effectuate a successful treatment.

In one embodiment, a diode laser of the invention is a high powered semiconductor laser. In one embodiment, the source of radiation is a fiber coupled diode laser array. For example, an optical source of radiation can include a plurality of light sources (e.g., semiconductor laser diodes) each adapted to emit a beam of light from a surface thereof. A plurality of first optical fibers each can have one end thereof adjacent the light emitting surface of a separate one of the light sources so as to receive the beam of light emitted therefrom. The other ends of the first optical fibers can be bundled together in closely spaced relation so as to effectively emit a single beam of light, which is a combination of the beams from all of the first optical fibers. A second optical fiber can have an end adjacent the other ends of the first optical fibers to receive the beam of light emitted from the bundle of first optical fibers. The beam of light from the bundled other ends of the first optical fibers can be directed into the second optical fiber. The first optical fiber can have a numerical aperture less than that of the second fiber.

In various embodiments, beams from multiple diode lasers or diode laser bars can be combined using one or more lens. In one embodiment, an array of diode lasers is mounted in a handpiece of the delivery system, and respective beams of radiation from each diode laser can be directed to the target region. The beams of radiation can be combined so that they are incident at substantially the same point. In one embodiment, the one or more lens direct the multiple beams of radiation into a single optical fiber. A handpiece of the delivery system projects the combined beam of radiation to the target region of skin.

The time duration of the cooling and of the radiation application can be adjusted so as to maximize the thermal injury to the vicinity of the dermal interface 142, as shown in FIG. 3. For example, if the position of the gland is known (e.g., by ultrasound imaging), then parameters of the optical radiation, such as pulse duration and/or fluence, can be optimized for a particular treatment. Cooling parameters, such as cooling time and/or delay between a cooling and irradiation, can also be optimized for a particular treatment. In some embodiments, in tissue where the dermal interface 142 is deeply situated, the cooling time can be lengthened such that cooling can be extended deeper into the skin. At the same time, the time duration of radiation application can be lengthened such that heat generated by the radiation in the region of dermis closer to the skin surface can be removed via thermal conduction and blood flow, thereby minimizing injury to the tissue overlying the dermal interface 142. Similarly if the dermis overlying the dermal interface 142 is thin, the time duration of cooling and of radiation application can be adjusted to be shorter, such that thermal injury is confined to the region proximate to the dermal interface 142. Accordingly, a zone of thermal treatment can be predetermined and/or controlled based on parameters selected. For example, the zone of thermal injury can be positioned in, at, or proximate to the dermal interface 142.

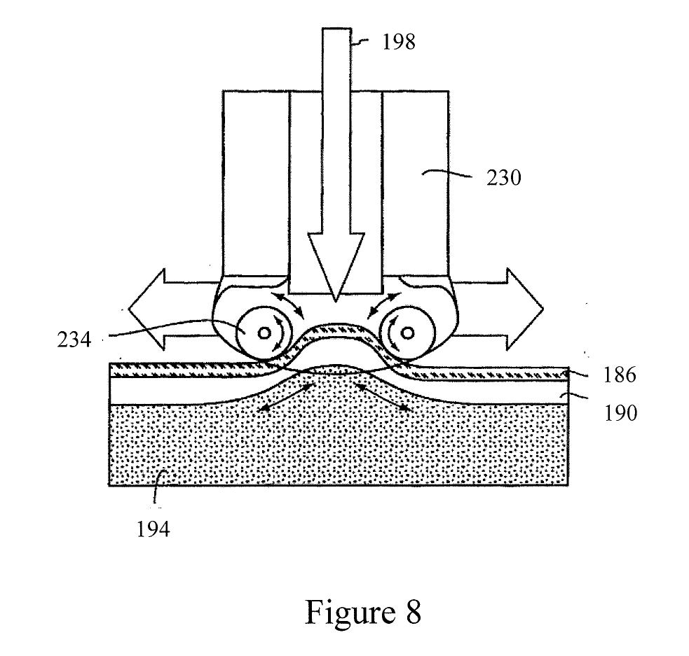

In various embodiments, the target region of skin can be massaged before, during, and/or after irradiation of the target region of skin. The massage can be a mechanical massage or can be manual massage. FIG. 8 depicts a handpiece 230 that includes rollers 234 to massage the skin. Radiation 198 can be delivered through a central portion of the handpiece 230. The massage handpiece 230 can be adapted to fit over the delivery system 162 shown in FIG. 4. In one embodiment, a delivery system can be formed with a mechanical massage device affixed. In one embodiment, vacuum can be used to pull the tissue into the device, which can provide an additional massage effect. In one embodiment, a person massages the target region of skin after irradiation of the tissue. Massaging the target region of skin can facilitate removal of treated glandular tissue from the target region. For example, massaging can facilitate draining from the treated region.

Functions of a sweat gland can be affected by targeting the gland after it is stained with an excitable material. The excitable material can be delivered to the target gland via a sweat duct connected to the gland and/or by absorption of the excitable material through the surface of the skin. A beam of radiation can be used to cause thermal injury to the gland through a surface of the epidermal layer by photodynamically or photothermally activating the excitable material. In particular, to cause photothermal injury to a target gland using a beam of radiation applied through a surface of the epidermal layer, the excitable material in the gland is adapted to absorb the beam of radiation and generate heat that injures, damages and/or destroys the surrounding cells. To cause photodynamic injury to a target gland using a beam of radiation, the excitable material in the gland is adapted to absorb the beam of radiation, generate reactive chemical species such as singlet oxygen which can in turn injure, damage or destroy the surrounding cells.

The excitable material can be topically applied to a surface of the epidermal layer. The material can also be delivered to the target gland by injection, massage, ultrasound, iontophoresis, or other means of transdermal delivery of compounds. Iontophoresis is a non-invasive technique that uses a repulsive electromotive force to propel a charged agent through the skin surface.

Exemplary excitable materials include dyes, colorants and chromophores. The excitable material can be a solution or a suspension. If the excitable material is a charged agent, the agent can be in a solution that is topically applied to a skin surface before being delivered to the gland using iontophoresis. The chromophore can be non-toxic and/or hydrophilic.

The wavelength of the beam of radiation can be selected to promote skin tissue penetration and/or be absorbed by the excitable material. In some embodiments, the excitable material is a type of chromophore that enhances light absorption, such as methylene blue, reactive black, or indocyanine green. The corresponding wavelength can be about 700 nm to about 800 nm. In some embodiments, the excitable material is a chromophore that is photodynamic in nature, in which case the wavelength can be about 630 nm.

The invention features a kit suitable for use in the treatment. The kit can be used to improve the cosmetic appearance of a region of skin. The kit can include a source of a beam of radiation and instruction means. The instruction means can include instructions for directing the beam of radiation to a gland. A cooling system can be used to cool an epidermal region or a dermal region of the target region to minimize substantial unwanted injury thereto. The instruction means can prescribe a wavelength, fluence, and/or pulse duration for treatment. The instruction means, e.g., treatment guidelines, can be provided in paper form, for example, as a leaflet, booklet, book, manual, or other like, or in electronic form, e.g., as a file recorded on a computer readable medium such as a drive, CD-ROM, DVD, or the like.

In some embodiments, the instruction means can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site.

The instruction means can be performed by one or more programmable processors executing a computer program to perform functions of the invention by operating on input data and generating output. The instruction means can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks.

The computing system can include clients and servers. A client and a server are generally remote from each other and typically interact through a communication network. The relationship of client and server arises by virtue of computer programs running on the respective computers and having a client-server relationship to each other.

While the invention has been particularly shown and described with reference to specific illustrative embodiments, it should be understood that various changes in form and detail may be made without departing from the spirit and scope of the invention.

What is claimed is:

1. A method of treating sweat glands, comprising:
generating electromagnetic radiation having a wavelength of about 700 nm to about 1,800 nm;
delivering the electromagnetic radiation to a dermal interface defined by a dermal region and a subcutaneous fat region in a target region of skin;
cooling an epidermal region of the skin at least one of before, during or after delivering the electromagnetic radiation to the dermal interface in the target region of skin; and
causing thermal injury to at least one of the dermal region, the subcutaneous fat region or the dermal interface to decrease sweat production in a plurality of sweat glands.

2. The method of claim 1 further comprising delivering the electromagnetic radiation to the target region to thermally injure the plurality of sweat gland.

3. The method of claim 1 further comprising delivering the electromagnetic radiation to the target region to thermally injure hair bulbs in communication with at least some of the sweat glands.

4. The method of claim 1 further comprising destroying at least some of the sweat glands.

5. The method of claim 1 wherein the electromagnetic radiation has a wavelength of about 1,190 nm to about 1,230 nm.

6. The method of claim 1 wherein the electromagnetic radiation has a wavelength of about 1,210 nm.

7. The method of claim 1 wherein the electromagnetic radiation has a wavelength of about 1,064 nm.

8. The method of claim 1 further comprising delivering the electromagnetic radiation to the target region about 0.5 mm to about 5 mm below the surface of the skin.

9. The method of claim 1 wherein the electromagnetic radiation has a fluence of about 30 J/cm² to about 300 J/cm².

10. The method of claim 1 wherein the electromagnetic radiation has a fluence of about 50 J/cm² to about 60 J/cm².

11. The method of claim 1 wherein the electromagnetic radiation has a pulse duration of about 1 second to about 10 seconds.

12. The method of claim 1 further comprising delivering the electromagnetic radiation to cause treatment temperature to peak at the dermal interface.

13. The method of claim 1 further comprising causing thermal injury to induce fibrosis in at least one of the dermal region, the subcutaneous fat region, or the dermal interface.

14. A method of treating sweat glands, comprising:
generating electromagnetic radiation having a wavelength of about 700 nm to about 1,800 nm;
delivering the electromagnetic radiation to hair bulbs in communication with at least some of the sweat glands in a target region of skin to cause thermal injury to the hair bulbs, thereby inducing decreased sweat production in the at least some of the sweat glands; and
cooling an epidermal region of the skin at least one of before, during or after delivering the electromagnetic radiation to the dermal interface in the target region of skin.

15. The method of claim 13 further comprising destroying the at least some of the sweat glands.

16. The method of claim 14 wherein the electromagnetic radiation has a wavelength of about 1,190 nm to about 1,230 nm.

17. The method of claim 14 wherein the electromagnetic radiation has a wavelength of about 1,210 nm.

18. The method of claim 14 wherein the electromagnetic radiation has a wavelength of about 1,064 nm.

19. An apparatus for treating sweat glands, comprising:
a source generating electromagnetic radiation having a wavelength of about 700 nm to about 1,800 nm;
a delivery system coupled to the source for directing the electromagnetic radiation to a dermal interface defined by a dermal region and a subcutaneous fat region in a target region of skin; and
a cooling system to cool an epidermal region of the skin at least one of before, during or after delivering the electromagnetic radiation to the dermal interface in the target region of skin;
wherein the electromagnetic radiation is adapted to cause thermal injury to at least one of the dermal region, the subcutaneous fat region or the dermal interface to decrease sweat production in a plurality of sweat glands.

20. The apparatus of claim 19 wherein the electromagnetic radiation has a wavelength of about 1,190 nm to about 1,230 nm.

21. The apparatus of claim 19 wherein the electromagnetic radiation has a wavelength of about 1,210 nm.

22. The apparatus of claim 19 wherein the electromagnetic radiation has a wavelength of about 1,064 nm.

23. The apparatus of claim 19 wherein the electromagnetic radiation has a fluence of about 1 J/cm² to about 500 J/cm².

24. The apparatus of claim 19 wherein the electromagnetic radiation has a pulse duration of about 1 second to about 10 seconds.
